(19) United States
(12) Patent Application Publication
(10) Pub. No.: US 2015/0346223 A1
(43) Pub. Date: Dec. 3, 2015

(54) FT4 ELECTROCHEMICAL ASSAY DETECTION SYSTEM

(71) Applicant: VANTIX HOLDINGS LIMITED, St. Helier (JE)

(72) Inventor: Andy Gover, Cambridgeshire (GB)

(21) Appl. No.: 14/759,759

(22) PCT Filed: Jan. 9, 2014
(86) PCT No.: PCT/GB2014/050056

§ 371 Date: Jul. 8, 2015

Related U.S. Application Data
(60) Provisional application No. 61/750,512, filed on Jan. 9, 2013.

(51) Int. Cl. G01N 33/78

(52) U.S. Cl. CPC G01N 33/78

(57) ABSTRACT

Some embodiments provide a method for performing a free thyroxin assay. The method may include providing a flow cell that may include a plurality of sensors at least partially coated with a plurality of antibodies that bind to free thyroxin. The method may further provide introducing a sample into the flow cell so that the sample contacts the sensors, and incubating the sample therein for a time. As a result, at least a portion of any free thyroxin within the sample can bind at least some of the antibodies. In addition the method may include introducing a wash agent into the flow cell to clear the flow cell of any substantial amount of the sample.

### CROSS-REFERENCE

The present disclosure claims priority to U.S. Provisional Application Ser. No. 61/750,512 filed Jan. 9, 2013, and entitled “Electrochemical Detection System,” which is incorporated herein by reference in its entirety.

### FIELD

The present disclosure generally relates to an electrochemical detection system for conducting electrochemical analysis, and more particularly to an electrochemical detection system that can be configured to perform multiple assays, including the detection of free thyroxin from undiluted and non-dialyzed whole blood.

### BACKGROUND

Electrochemistry is a branch of chemistry that studies chemical reactions occurring in a solution at the interface of an electron conductor and an electrolyte. The reaction may involve electron transfer between the electron conductor and the electrolyte. For example, the electron conductor may be an electrode comprising a metal or a semiconductor.

Under some circumstances, applying either an externally derived voltage or a voltage created by the chemical reaction may drive the chemical reactions discussed above. Under these circumstances, these chemical reactions are known as electrochemical reactions. Moreover, some chemical reactions where electrons are transferred between one or more molecules are known as oxidation/reduction or redox reactions. Generally, electrochemistry relates to situations where oxidation and reduction reactions are separated in space or time and are connected by an external electric circuit that may be used to understand the reaction.

Some electrochemical analyses may be undertaken in a disposable cartridge including a reagent for inducing electrochemical reactions monitored, detected, or quantified by one or more sensors. Some conventional cartridges may be configured to operatively engage a reader device that initiates a protocol, such as via mechanical actuation of the cartridge. Furthermore, the reader device may receive data signals that may be processed to produce test results of the reaction occurring within the cartridge.

By way of example, some conventional cartridges may be used in conjunction with immunoassays. For example, a portion of the conventional cartridge may be coated in one or more types of antibodies that have been designed to bind to a preselected target molecule or compound. As a result, the target molecule or compound may bind to the antibodies and different methods of quantification may be used to assess the quantity of the target within a sample.

Accurate and rapid measurements of free thyroxin, a thyroid hormone also known as FT4, may be used to differentiate between primary and second hypothyroidism and to diagnose other potential medical conditions. Conventional methods of measuring free thyroxin circulating in a patient's blood may be difficult and time consuming For example, conventional methods include equilibrium dialysis that may take several hours at significant cost.

### SUMMARY

Accordingly, a system is provided whereby free thyroxin levels can be readily assessed in a quick and accurate manner at the point of care. In one embodiment, the electrochemical detection system includes a method for performing a free thyroxin assay. In some embodiments, the method may include providing a flow cell, which includes a plurality of sensors that may be at least partially coated with a plurality of antibodies that bind to only free thyroxin; that is, thyroxin not bound to one or more serum binding proteins. The method may further provide introducing a sample into the flow cell so that the sample contacts the plurality of sensors, and incubating the sample within the flow cell for a time, such as less than or equal to two minutes. As a result of the incubation, at least a portion of free thyroxin contained within the sample may bind to at least some of the antibodies coupled to the sensors. In addition, the method may include introducing a wash agent into the flow cell to clear the flow cell of any substantial amounts of the sample. For example, in some embodiments, the sample may be undiluted or non-dialyzed whole blood and the wash agent may be a liquid, such as water, an aqueous solution, an alcohol, for example ethanol or methanol, acetonitrile, dimethylformamide (DMF), dimethylsulfoxane (DMSO), or other polar solvent; or a gas, such as air, nitrogen, argon, helium, or oxygen.

In one aspect, the method may further provide introducing a solution into the flow cell after introducing the wash agent. For example, the solution may be a solution of known amounts of thyroxin that have been conjugated to an enzyme, such as horseradish peroxidase. As a result of the addition of the solution to the flow cell, at least a portion of the thyroxin within the solution may bind to antibodies that did not previously bind to thyroxin within the sample.

In one aspect, after adding the solution, another volume of the wash agent may be introduced into the flow cell to clear the flow cell of any substantial amount of the solution. In some embodiments, after washing of the flow cell, a substrate may be introduced into the flow cell so that the substrate contacts the plurality of sensors. Thereafter, the substrate may be incubated within the flow cell so that the substrate is processed by the enzyme conjugated to the thyroxin bound to the antibodies.

In some embodiments, the electrochemical detection system includes a method for manufacturing a platform that can perform at least a free thyroxin assay. For example, the method may include providing an input port that can be in selective fluid flow communication with at least one flow cell. The method may further provide disposing a plurality of sensors with the at least one flow cell and coating at least a portion of the plurality of sensors with a plurality of antibodies that can bind to free thyroxin found in undiluted and non-dialyzed whole blood. In addition, the method may include providing a plurality of reservoirs such that at least some of the plurality of reservoirs contains a wash agent (for example a fluid such as a liquid or air), thyroxin conjugated to an enzyme (e.g., horseradish peroxidase), and a substrate. In some embodiments, the method can further include providing a plurality of channels that may fluidly connect the flow cell, the input port, and the plurality of reservoirs. In some embodiments, the method may further include at least partially coating the plurality of sensors with antibodies that can bind to other molecules, such as thyroid-stimulating hormone or other forms of thyroxin.

In yet another aspect, the electrochemical detection system may include a method for performing a free thyroxin assay. In some embodiments, the method may include providing a flow cell, which includes a plurality of sensors that may be at least partially coated with a plurality of antibodies that bind to free thyroxin. The method may further provide introducing a volume of undiluted and/or non-dialyzed whole blood into the flow cell so that the whole blood contacts the plurality of sensors. The method further provides incubating the undiluted whole blood within the flow cells for a time. As a result of the incubation, at least a portion of any free thyroxin contained within the whole blood can bind to at least some antibodies coupled to the sensor. In addition, the method may include introducing a first volume of a wash agent into the flow cell to clear the flow cell of any substantial amounts of the whole blood.

In one aspect, the method may further include introducing a solution into the flow cell after the washing. Thereafter, the solution may be incubated within the flow cell for a time. For example, the solution may include thyroxin conjugated to an enzyme. As a result, the thyroxin contained within the solution can bind to at least some antibodies not bound to the thyroxin contained within the whole blood. The method then may provide introducing a second volume of the wash agent into the flow cell to clear the flow cell of any substantial amounts of the solution, introducing a substrate into the flow cell, and incubating the substrate within the flow cell. In some embodiments, the method may further provide introducing a third volume of the wash agent into the flow cell to clear the flow cell of any substantial amounts of the substrate, applying a voltage to the sensors, and reading the sensors to complete the assay.

In one aspect, the electrochemical detection system may include a platform for performing a free thyroxin assay. For example, the platform may include a plurality of sensors at least partially disposed within a flow cell. Moreover, the sensors may be at least partially coated with a plurality of antibodies that can bind free thyroxin. In addition, the platform may include a first, a second, a third, and a fourth fluid source in selective fluid flow communication with the flow cell. For example the first fluid source may be able to contain a volume of undiluted and non-dialyzed whole blood. The second fluid source may contain a wash reagent, such as air or other fluids. The third fluid source may contain a solution that includes thyroxin previously conjugated to an enzyme. And, the fourth fluid source may contain a substrate that can be recognized by the enzyme conjugated to the thyroxin.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is simplified block diagram illustrating the different components of the electrochemical detection system.

FIG. 2 is a simplified illustration showing the cartridge and reader arrangement.

FIG. 3 is a top view of the sensor arrangement used in the cartridge for electrochemical detection.

FIG. 4 is a top view of another embodiment of the sensor arrangement used in the cartridge for electrochemical detection.

FIG. 5 is an exploded view of the sensor arrangement shown in FIG. 3.

FIG. 6 is a plan view of the sensor operatively engaged to the flow cell.

FIG. 7 is a line graph detailing results of an experiment testing an embodiment of the disclosure.

Corresponding reference characters indicate corresponding elements among the view of the drawings. The headings used in the figures should not be interpreted to limit the scope of the claims.

### DETAILED DESCRIPTION

An accurate measurement of circulating free thyroxin in a patient's blood is a relevant component of diagnosing a thyroid disease. In particular, free thyroxin, otherwise known as FT4, is used to differentiate between primary and secondary hypothyroidism, an increasingly common condition worldwide. In most humans, the majority of thyroxin in the blood circulates bound to serum-binding proteins, such as thyroxin-binding globulin, albumin, or pre-albumin. Of the total concentration of thyroxin in relatively healthy individual, approximately 0.03% of thyroxin is unbound or “free.” The concentration of the free or unbound thyroxin is metabolically relevant and provides diagnostic utility over a total thyroxin concentration.

Free thyroxin exists in equilibrium with bound thyroxin, which complicates measuring the concentration. In particular, one difficult aspect of obtaining accurate measurements of free thyroxin is needing to not significantly disrupt the equilibrium between bound and unbound thyroxin. If this equilibrium is disturbed, inaccurate measurements may occur.

At least some of the current conventional methods for measuring free thyroxin include equilibrium dialysis, which uses a dialysis membrane. Over the several hours (typically 17 hours), a blood sample from a patient may be dialyzed across a dialysis membrane to equilibrate the sample. As a result, little to no free thyroxin is sequestered, ensuring that anomalous serum-binding protein levels or affinities do not affect this method. However, the extensive time to produce these results delays proper diagnoses.

Some conventional “rapid” assays are available, but these have their own drawbacks. Specifically, some conventional rapid assays use a dilution at the first step of the assay, and the diluted sample is exposed to an anti-thyroxin antibody. As the anti-thyroxin antibodies bind the free thyroxin, bound thyroxin is released, thereby altering the natural equilibrium between bound and unbound thyroxin. Moreover, only limited dilutions can be achieved because as the sample is further diluted, the ratio of antibody to binding protein is increased. As this ratio increases, it can be more difficult to accurately determine the concentrations of free thyroxin.

Accordingly provided herein is an electrochemical detection system that enables the detection of free thyroxin without dialysis or dilution. For example, in some embodiments, the electrochemical detection system may have at least one flow cell with a plurality of sensors. The sensors may be coated with one or more antibodies or other molecules that can bind free thyroxin. For example, the plurality of sensors may be coated with an anti-thyroxin antibody that only binds free thyroxin; that is, the antibodies will not bind thyroxin bound to serum-binding proteins.

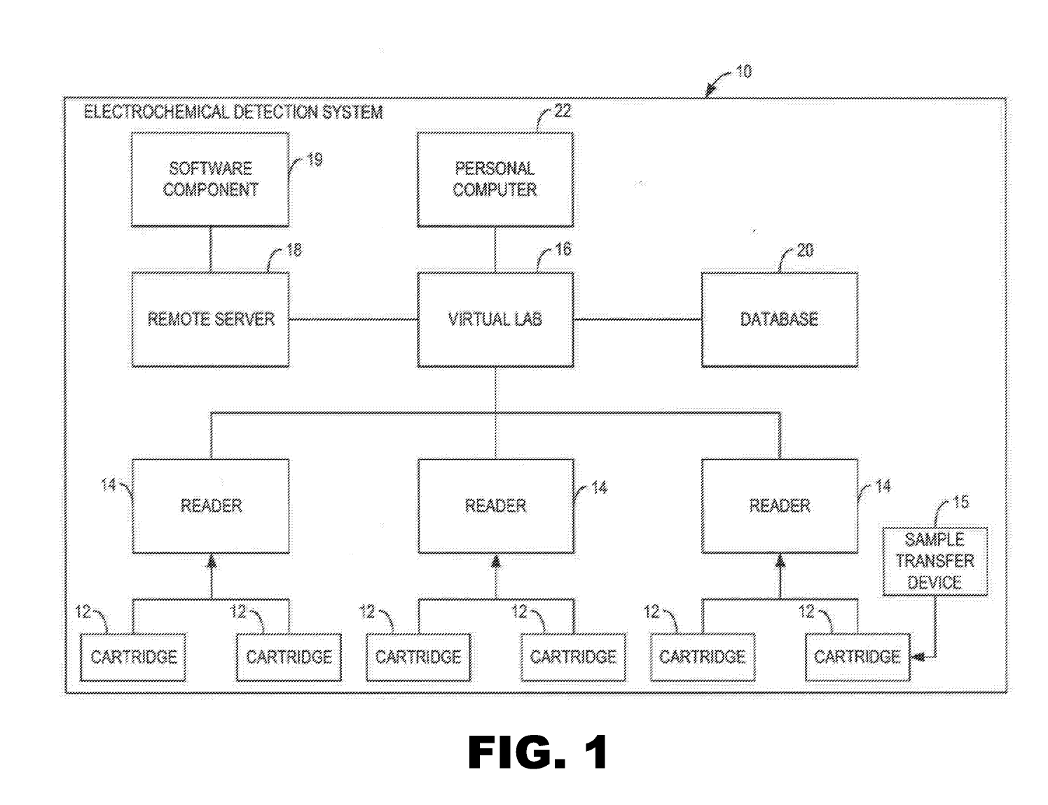

Referring to the drawings, an embodiment of the electrochemical detection system is illustrated and generally indicated as 10 in FIG. 1. The electrochemical detection system 10 provides a means for conducting a plurality of assay protocols on a single disposable cartridge 12 when operatively engaged to a reader 14. In addition, the electrochemical detection system 10 may include a plurality of readers 14 in operative communication with a virtual lab 16 for communicating data, such as test results or calibration information, between the readers 14 and a remote server 18 associated with the virtual lab 16. As mentioned above, the electrochemical detection system 10 may be used to conduct or perform a plurality of assay protocols, such as assay protocols for detecting one or more molecules found in the blood of an animal, such as a human or companion animal, for example a dog or cat. In some embodiments, the electrochemical detection system 10 may be configured and arranged for use in conducting an assay to detect the concentration of thyroxin within a blood sample. For example, the electrochemical detection system 10 may be configured to detect levels of free thyroxin in substantially or completely undiluted or non-dialyzed whole blood samples.

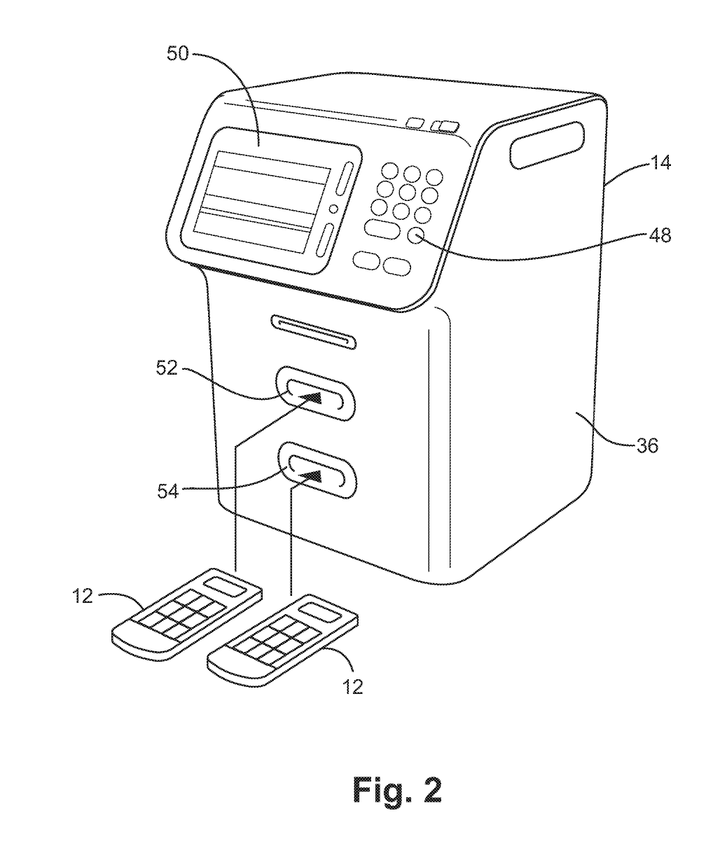

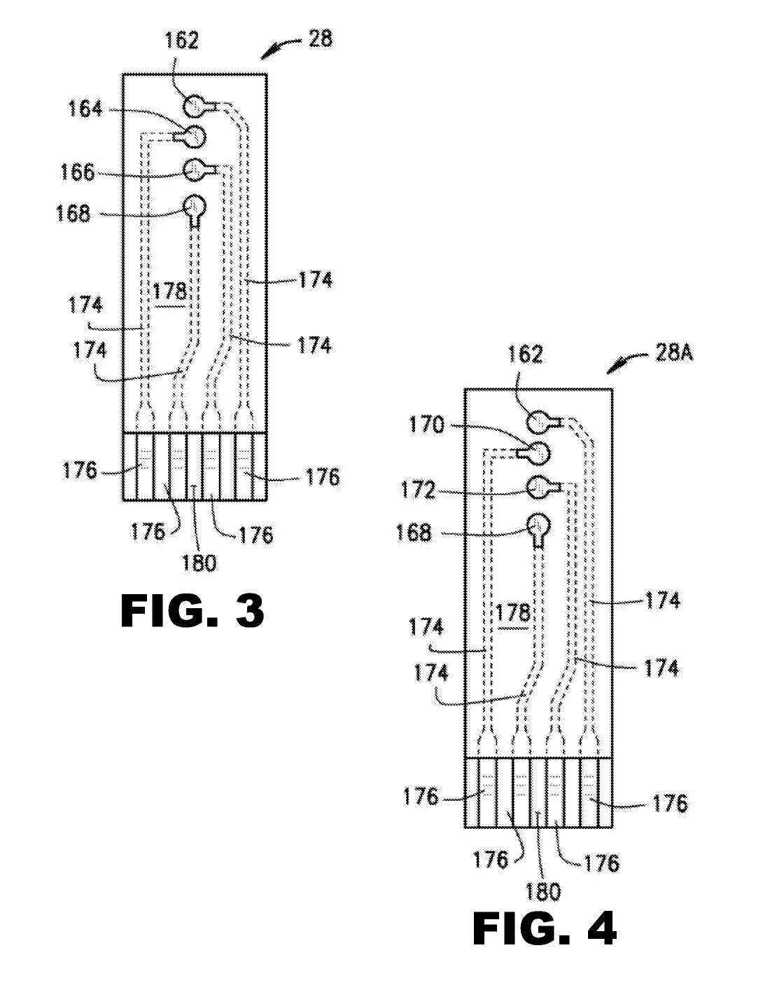

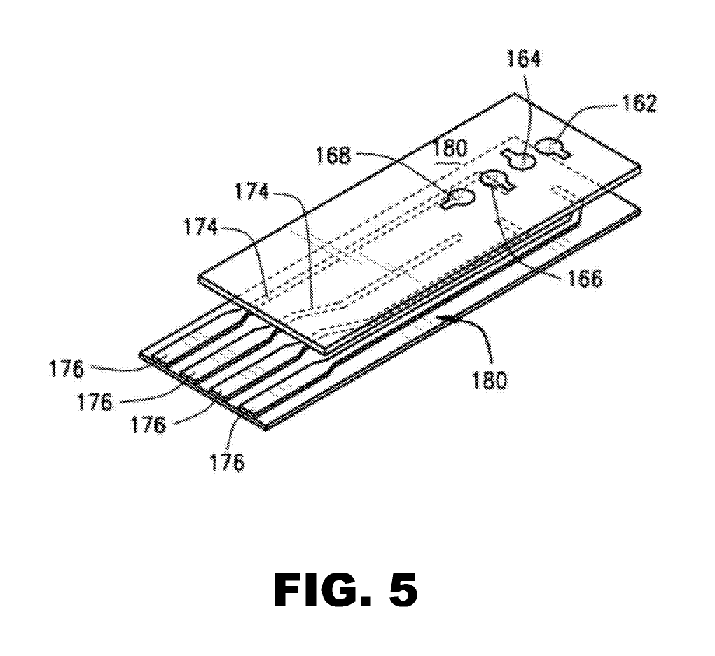

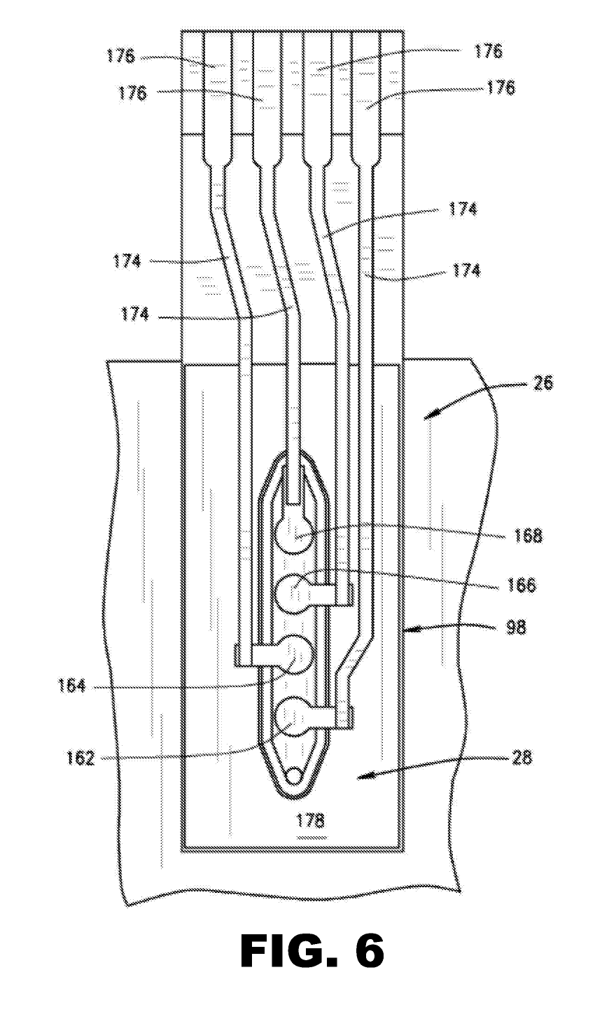

As shown in FIG. 2, each reader 14 may include a reader body 36 having a control panel 48 that permits the user to perform a plurality of assay protocols when a respective cartridge 12 is operatively engaged to the reader 14. In one embodiment, the reader 14 may include a first docking station 52 and a second docking station 54 for operative engagement of a respective cartridge 12 with the reader 14. Other embodiments of the reader 14 may include one or more docking stations for engaging any number of respective cartridges 12. The reader 14 further includes a screen 50 that acts as a user interface and a communication component (not shown), permitting the reader 14 to operatively communicate with the virtual lab 16 through the remote server 18.

In one aspect, the electrochemical detection system 10 can rapidly perform immunoassay reactions within cartridge 12 by the sequentially controlled release of fluids through the flow cell chambers 94A, 96A, and 98A when the assay protocols are conducted.

Based on reaction kinetics and thermodynamics, higher concentrations of these molecules in the same vicinity result in faster reaction rates. For example, in a typical immunoassay, reagents may be consecutively pipetted into a microtiter tray cuvette containing antibodies attached to the surface of the cuvette. The target analyte binds to the antibody in a chemical reaction. In this format, chemical reactions can take 30-40 minutes before detection is possible. In the microtiter tray format in particular, antibodies attached to the surface of the cuvette react with the target analyte molecules.

As target molecules closest to the antibodies bind or react with the antibodies, the region closest to the antibodies inevitably depletes the target molecules. The diffusion of new target molecules replenishes the area to permit further binding. This type of diffusion, however, is a slow, limited reaction-type system and uses more time for binding. Similarly, as other reagents are added that also interact with the antibodies, the rate of diffusion in the bulk solution controls the rate of the reactions. One method is to agitate the bulk solution in the cuvette using a stirring bar or by agitating the microtiter tray itself. This agitating action refreshes the region closest to the antibodies that provides new target molecules.

Microfluidic platforms, such as disposable cartridges, can only store small volumes of fluid compared to the microtiter tray format; however, rapid immunoassay reactions are still achievable using flow cells 94, 96, and 98. The flow cell format of cartridge 12 allows the surface area closest to the antibodies to be replenished by sequentially flowing liquids, such as reagents, through flow cells 94, 96, and 98, thereby achieving the agitation noted above. In the alternative, cartridge 12 may be able to flow a small block of liquid, such as reagent, back and forth over the surface closest to the antibodies. This alternative arrangement provides the same replenishing action using less reagent and sample. As such, both arrangements rapidly redistribute the target analyte and reagents evenly throughout the solution, thereby preventing the reactions from becoming diffusion limited.

Some embodiments of the electrochemical detection system 10 may operate in a generally similar manner to some previously mentioned embodiments, but with different physical configurations. In some embodiments, in lieu of the previously mentioned cartridge 12, the electrochemical detection system 10 may operate using a general platform, including a support structure. For example, the support structure may function in a substantially similar manner to the fluidics backbone 26. The support structure may use different configurations than discussed above. Accordingly, no embodiment discussed above or below requires the use of a fluidics backbone 26 of the configuration discussed above. Rather, some embodiments of the electrochemical detection system 10 include a support structure with other configurations to support one or more flow cells that may each comprise a plurality of sensors.

In some exemplary embodiments, the sensors may be coated with a material such as streptavidin and the antibodies may be biotinylated, to retain the antibodies on the sensors. In other embodiments, electrostatic interactions between the antibodies and sensors may retain the antibodies. In some embodiments, the plurality of sensors discussed above may be coated with different types of antibodies. For example, in one embodiment, at least one of the plurality of sensors may be at least partially coated with one or more antibodies that can bind free thyroxin. In addition, the same at least one plurality of sensors may also be at least partially coated in another antibody that can bind a different molecule, such a thyroid-stimulating hormone or a different form of thyroxin.

In one embodiment, after the plurality of sensors is at least partially coated, a sample may be introduced into the flow cell and allowed to contact the plurality of sensors. For example, the sample may be incubated in the flow cell for a time. In some embodiments, the time may be less than or equal to two minutes. In other embodiments, the time may be any period to achieve the desired results. As the sample is exposed to the sensor and the antibodies, at least a portion of any free thyroxin contained within sample can bind to the anti-thyroxin antibodies bound thereto.

In some embodiments, the sample may be undiluted whole blood. Because of the sensitivity of the sensors, the antibody, and the relatively short time that the sample is exposed to the sensors, dialysis or dilution of the sample may not be needed before testing, as opposed to conventional systems. Nevertheless, the whole blood sample may be dialyzed or diluted before the thyroxin assay is run according to the disclosure.

After the samples are exposed to the sensors and the antibodies bound thereto, the excess sample may be washed from the flow cell. As described above, one of several different washing reagents may be used to clear the flow cell. For example, air may be passed through the flow cell and over the sensors to remove the sample from the flow cell, because of the laminar flow of the air through the flow cell. In other embodiments, other fluids, such as liquids, may be used to wash the flow cell and sensors. Suitable liquids include, but are not limited to, water, an aqueous solution, such as a buffer, for example phosphate buffered saline (PBS); an alcohol, for example ethanol or methanol; acetonitrile, dimethylformamide (DMF), dimethylsulfoxane (DMSO), and other polar solvents. As a result of washing, largely only free thyroxin bound to the anti-thyroxin antibodies remains in the flow cell.

In one embodiment, after the sample has passed through the flow cell and over the sensors, a solution may then be passed through the flow cell. For example, the solution may include free thyroxin that has been previously conjugated to an enzyme. Suitible enzymes include, but are not limited to, peroxidase, laccase, oxidase, catalase, urease, kinase, dehydrogenase, and deiminase. Suitable peroxidases include, but are not limited to, horseradish peroxidase (HRP), deiodinase, such as iodothyronine diodinase and iodotyrosine deiodinase; eosinophil peroxidase, glutathione peroxidase, such as GPX 1, GPX 2, GPX 3, GPX 4, GPX 5, GPX 6, GPX 7, and GPX 8; haloperoxidase, myeloperoxidate (MPO), hemoprotein, peroxiredoxin, thyroid peroxidase, vanadium bromoperoxidase, and lactoperoxidase. Suitable oxidases include, but are not limited to, laccase, glucose oxidase, monoamine oxidate (MAO), cyctochrome P450 oxidase, NADPH oxidase, xanthine oxidase, L-gulonolactone oxidase, and lysyl oxidase. Suitable kinases (also referred to as phosphotransferases) include, but are not limited to, OH acceptor kinases, such as hexokinase, glucokinase, fructokinase, hepatic fructokinase, galactokinase, phosphofructokinase 1, phosphofructokinase liver-type (PFKL), phosphofructokinase muscle-type (PFKM), phosphofructokinase platelet-type (PFKP), phosphofructokinase 2, riboflavin kinase, shikimate kinase, thymidine kinase, ADP-thymidine kinase, NAD+ kinase, glycerol kinase, pantothenate kinase, mevalonate kinase, pyruvate kinase, deoxycytidine kinase, fructose-6-phosphate 1-phosphotransferase (PFP), diacylglycerol kinase, phosphoinositide 3 kinase (Class I PI 3, abd Class II PI 3), sphingosine kinase, and glucose-1,6-bisphosphate synthase; COOH acceptor kinases, such as phosphoglycerate aspartate kinase; N acceptor kinases such as creatine; PO₄ acceptor kinases, such as phosphomevalonate kinase, adenylate kinase, nucleoside-diphosphate kinase, uridylate kinase, guanylate kinase, and thiamine-diphosphate kinase; and diphosphotransferases (P₂O₇), such as ribose-phosphate diphosphokinase and thiamine diphosphokinase. Suitable dehydrogenases include, but are not limited to, aldehyde dehydrogenase acetaldehyde dehydrogenase, alcohol dehydrogenase, glutamate dehydrogenase, lactate dehydrogenase, pyruvate dehydrogenase, glucose-6-phosphate dehydrogenase, glyceraldehyde-3-phosphate dehydrogenase, sorbitol dehydrogenase, isocitrate dehydrogenase, alpha-ketoglutarate dehydrogenase, succinate dehydrogenase, ad malate dehydrogenase. Suitable deiminases include, but are not limited to, arginine deiminase. In exemplary embodiments, the enzyme is a peroxidase, such as horseradish peroxidase. In other exemplary embodiments, the enzyme is a laccase or a catalase.

As the solution passes through the flow cell and over the sensors, the free thyroxin within the solution may bind to the anti-thyroxin antibodies that did not bind to free thyroxin within the sample. In particular, this binding is generally similar to competitive binding assay. After the solution passes through the flow cell and over the sensors, the wash step may be repeated to remove any significant amount of the solution from within the flow cell. In some embodiments, the wash step may be performed with the same or a different washing reagent.

After the second wash step, a substrate may be passed through the flow cell. Specifically, the substrate may be chosen in accord with the enzyme conjugated to the thyroxin in the solution. For example, in the case of horseradish peroxidase, materials such as Lumigen® substrate may be used. In this step, the substrate may be cleaved by the enzyme bound to the sensors, such as via the thyroxin added in the solution, and the sensors may be used in accord with some previously mentioned embodiments. For example, the reaction of the substrate with the enzyme may generate a potential that can be detected by the sensors and data related to this potential may be sent to a component for calculations, for example the software component 19. Specifically, in the example discussed above, the amount of free thyroxin in the original sample may exhibit a generally inverse relationship relative to the amount of signal detected.

When introducing elements of the present disclosure or the embodiments(s) thereof, the articles “a,” “an,” “the,” and “said” are intended to mean that there are one or more of the elements. The terms “comprising,” “including,” and “having” are intended to be inclusive and mean that there may be additional elements other than the listed elements.

### EXAMPLES

The following examples detail some manners in which one skilled in the art can employ some embodiments of the electrochemical detection system 10. The following examples are not intended to be limiting of the disclosure and the claims, but rather an illustrative discussion regarding some uses of the system.

### Example 1

### Assay Protocol 1—Free T4—Typical Competitive Immunoassay

The conjugate was diluted 1:5000 in Stabilzyme® horseradish peroxidase (HRP) and the thyroxin (T4) antibody diluted in a phosphate buffered saline (PBS) to provide a final concentration of 1.0 μg/mL. A sample having a T4 antibody in a ratio of 25 μL antibody to 100 μL sample was provided to one of the flow cells 94, 96, or 98 followed by the wash buffer and a substrate. For example, PBS was used as the wash buffer, while the substrate was provided at a concentration of one set of SigmaFAST tablets added for every 50 mL of MilliQ water.

In one embodiment, the sample with the antibodies have a volume of 150 μL and a flow rate of 1 μL per second when positively displaced by the sample reservoir 46 to one or more flow cells 94, 96, and 98. Once the sample was displaced, 150 μL of tracer was provided at a flow rate of 1 μL per second followed by 400 μL of wash buffer at a flow rate of 3 μL per second. Finally, 200 μL of substrate was provided at a flow rate of 3 μL per second. The following flow times was used: free T4 sample at 2.5 minutes, conjugate at 2.5 minutes, wash buffer at 2.25 minutes, substrate at 1.25 minutes, and read time at 1 minute and 40 seconds. As such, the assay protocol was performed in less than 10 minutes.

The sensors 28 was read using an applied voltage of −115 mV for 10 seconds, allowing an open circuit potential (OCP) for 5 seconds, applying −115 mV for 8 seconds, allowing OCP for 8 seconds, re-applying −115 mV for 8 seconds, and allowing an OCP for 90 seconds. The mV reading at the end of the 90-second OCP was taken as the final value by the software component 19.

In another aspect of the electrochemical detection system, the reader 14 mechanically actuated the cartridge 12 from one side of the cartridge 12. This permited the reader 14 to provide a heating source (not shown) for thermally controlling the side opposite of the side mechanically actuated by the reader 14.

### Example 2

### Assay Protocol 2—Free Thyroxin Assay

In this example, the sensors 28 were positioned or otherwise disposed within simple flow cells. Initially, the anti-thyroxin antibody (clone 1H1) was coated onto the sensors 28. Specifically, a solution of 0.1 μg/mL anti-thyroxin antibody was prepared in 0.05 M potassium phosphate (pH 8.0) and coated onto the sensors 28. The coated sensors 28 were stabilized with StabilGuard®. In addition, thyroxin that had been previously conjugated to horseradish peroxidase (the conjugate) was also diluted at a ratio of 1:40000 in a conjugate buffer.

After these initial steps, the samples were added to the sensors 28. Specifically, the first sample was the negative control with plasma that had all thyroxin stripped from it. The second sample contained a known concentration of thyroxin of 19 pmol, while the third sample contained a known concentration of thyroxin of 64 pmol. Each sample was added to individual flow cells that contained coated sensors 28.

Each of the three samples underwent the same procedure. In particular, about 60 μL of sample was aspirated at a rate of 0.5 μL per second for a total of 120 seconds to the sensors 28. Thereafter, air was passed over the sensors 28 to remove excess sample. After the air wash, the diluted conjugate was added to the sensors 28. The diluted conjugate was added to the coated sensors 28. About 60 μL of diluted conjugate was aspirated at a rate of 0.5 μL per second for 120 seconds. Thereafter, the air was passed over the sensors 28 to remove excess diluted conjugate. Next, a substrate was added to the sensors 28. For example, in this case, Lumigen® substrate was used according to manufacturer's instructions. About 50 μL of substrate was aspirated at a rate of 0.5 μL per second for 100 seconds. Thereafter, air was passed over the sensors 28 to remove excess substrate.

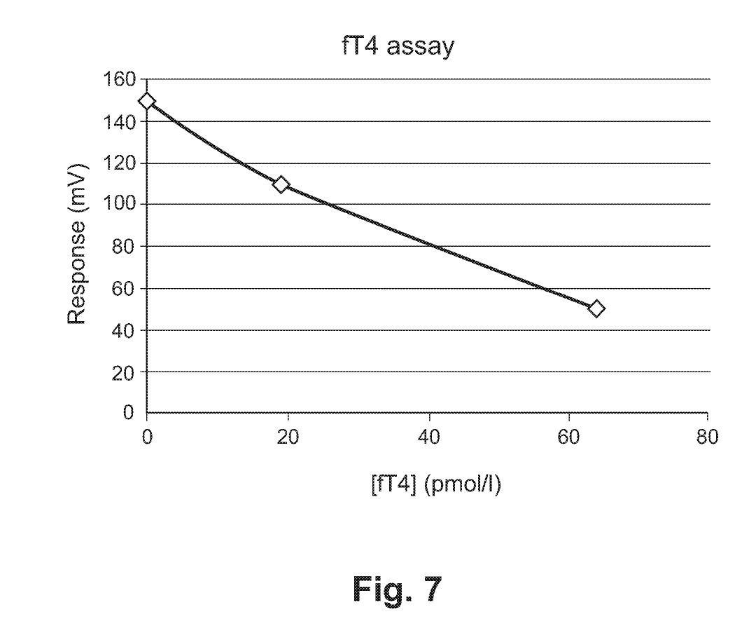

After adding the substrate, a voltage of −50 mV was applied to the sensors 28 for 5 seconds. After which, an additional 20 μL of substrate was aspirated at a rate of 0.5 μL per second for 40 seconds. Readings were taken, the data from which are shown in FIG. 7. Specifically, data obtained from the samples of known concentration were accurately correlated with the response in mV. Specifically, the greater the concentration of thyroxin within the sample, the lesser the voltage read at the sensors 28. For example, the plasma sample stripped of thyroxin exhibited a voltage of about 150 mV, while the second sample with the greatest concentration of thyroxin exhibited the lowest voltage of around 30 mV.

It should he understood from the foregoing that, while particular embodiments have been illustrated and described, various modifications can be made thereto without departing from the spirit and scope of the disclosure as will be apparent to those skilled in the art. Such changes and modifications are within the scope and teachings of this disclosure as defined in the claims appended hereto.

1. A method for performing a free thyroxin assay, the method comprising:
providing a flow cell including a plurality of sensors at least partially coated with a plurality of antibodies that can bind free thyroxin;
contacting a sample with the plurality of sensors;
incubating the sample within the flow cell for a time so that at least a portion of any free thyroxin contained within the sample binds at least some of the plurality of antibodies; and
introducing a wash agent into the flow cell to clear the flow cell of any substantial amount of the sample.

2. The method of claim 1, wherein the sample comprises undiluted whole blood.

3. The method of claim 1, further comprising introducing a solution into the flow cell after introducing the wash agent.

4. The method of claim 3, wherein the solution comprises thyroxin bound to an enzyme.

5. The method of claim 3, further comprising introducing a wash agent into the flow cell to clear the flow cell of any substantial amounts of the solution.

6. The method of claim 5, further comprising contacting a substrate with the plurality of sensors; and incubating the substrate within the flow cells.

7. The method of claim 1, wherein the time is lesser than or equal to two minutes.

8. The method of claim 1, wherein the wash agent is a liquid or air.

9. The method of claim 1, wherein the antibody is an anti-thyroxin antibody.

10. A method for manufacturing a platform that can perform at least a free thyroxin assay, the method comprising:
providing an input port in selective fluid flow communication with at least one flow cell;
disposing a plurality of sensors with the at least one flow cell;
coating at least a portion of the plurality of sensors with a plurality of antibodies that can bind free thyroxin; and
providing a plurality of reservoirs, wherein at least some plurality of reservoirs contains a wash agent, thyroxin conjugated to an enzyme, and a substrate.

11. The method of claim 10, further comprising providing a plurality of channels that fluidly connect the at least one flow cell, the input port, and the plurality of reservoirs.

12. The method of claim 10, wherein the enzyme is horseradish peroxidase.

13. The method of claim 10, wherein the wash agent is a fluid.

14. The method of claim 13, wherein the wash agent is at least one of air or a liquid.

15. A method for performing a free thyroxin assay, the method comprising:
providing a flow cell, the flow cell including a plurality of sensors at least partially coated with a plurality of antibodies that bind free thyroxin;
introducing a volume of undiluted whole blood into the flow cell so that the undiluted whole blood contacts the plurality of sensors;
incubating the undiluted whole blood within the flow cells for a time so that at least a portion of free thyroxin contained within the undiluted whole blood binds to at least some plurality of antibodies;
introducing a first volume of a wash agent into the flow cell to clear the flow cell of any substantial amount of the undiluted whole blood;
introducing into the flow cell a solution comprising thyroxin conjugated to an enzyme, wherein the solution is introduced into the flow cell so that the solution contacts the sensors;
incubating the solution within the flow cell for a time so that at least a portion of the thyroxin within the solution binds to at least a portion of the plurality of antibodies that have not already bound thyroxin from the undiluted whole blood;
introducing a second volume of a wash agent into the flow cell to clear the flow cell of any substantial amount of the solution; and
introducing a substrate into the flow cell.

16. The method of claim 15, further comprising incubating the substrate within the flow cell for a time.

17. The method of claim 16, further comprising applying a conditioning voltage to the sensors for a time and reading the sensors at a time after the voltage has been discontinued.

18. The method of claim 15, wherein the wash agent comprises a fluid.

19. The method of claim 18, wherein the fluid is one of a liquid and air.

20. A system for performing a free thyroxin assay, comprising:
a plurality of sensors at least partially disposed within a flow cell, wherein the plurality of sensors are at least partially coated with a plurality of antibodies that bind free thyroxin;
a first fluid source in selective fluid flow communication with the flow cell, wherein the first fluid source can contain a volume of undiluted and non-dialyzed whole blood;
a second fluid source in selective fluid flow communication with the flow cell, wherein the second fluid source contains a wash reagent;
a third fluid source in selective fluid flow communication with the flow cell, wherein the third fluid source contains a solution comprising thyroxin conjugated to an enzyme; and
a fourth fluid source in selective fluid flow communication with the flow cell, wherein the fourth fluid source contains a substrate recognized by the enzyme.
